(19) United States
(12) Patent Application Publication
(10) Pub. No.: US 2010/0191048 A1
(43) Pub. Date: Jul. 29, 2010

(54) NETWORK ENABLED SEXUAL APPARATUS WITH FEEDBACK AND FAULT TOLERANCE

(76) Inventor: Konstantin Kulikov, Rochester, MN (US)

Correspondence Address: PATWRITE LLC, 408 W. MAIN ST., MARSHALLTOWN, IA, 50158-5759, US

(21) Appl. No.: 12/689,975

(22) Filed: Jan. 19, 2010

Related U.S. Application Data
(60) Provisional application No. 61/145,281, filed on Jan. 16, 2009.

(51) Int. Cl. A61F 5/00; A61H 19/00

(52) U.S. Cl. 600/38; 601/49

(57) ABSTRACT

A network enabled sexual apparatus with feedback and fault tolerance comprises a saddle portion supported on a housing. A sexual stimulator attachment is moveably connected to a movement means which moves the sexual stimulator attachment. A control arm is moveably connected to the housing and is in communication with the sexual stimulator attachment and moves the sexual stimulator attachment in response to movements of the control arm. A control means remotely controls the network enabled sexual apparatus by being in communication with the control means. In one embodiment, a live signal is buffered and used to augment a background control function to overcome network lag. Another embodiment includes a display to allow a user to view remotes users and to interact with them.

### CROSS REFERENCE TO RELATED APPLICATIONS

This application claims priority and herein incorporates by reference U.S. provisional patent application 61/145,281, filed Jan. 16, 2009.

### BACKGROUND OF THE INVENTION

Throughout history sex has played an important role in shaping society, whether through completely hedonistic activity or Victorian repression, the pendulum of sexual expression swings back in forth in our history. Technology has been increasing in accordance with the so called Moore's law doubling processing power every 18 to 24 months with no foreseeable end to this trend in sight. Technological advances impact all aspects of human activity, so it is completely expected that the technological advances would also find expression in human sexuality.

The number of people joining the online community through the internet has exploded in the last decade with some parts of the world experiencing over 1000% increases and over 100% in the US. People shop, date, research, attend classes and play games through the world wide web and of course sexually oriented material is pervasive on the web. According to some studies, nearly 20% of all adult women (over 18) have visited a sexually explicit website and almost 50% for adult men.

Internet dating sites cater to all kinds of romantic interests and offer people a way to meet people in ways that were not available before the web. In addition to dating, there are many websites which encourage fantasy online relationships that often include cybersex which can include masturbation. The use of webcams increases the level of interaction in the cybersex experience, but for real sexual release, masturbation remains the key element in all these forms of cybersex. There is a need for web enabled appliance that can increase the interactivity of the sexual experience when using a network connection to experience sexual pleasure.

### SUMMARY OF THE INVENTION

A network enabled sexual apparatus with feedback and fault tolerance comprises a saddle portion supported on a housing. A sexual stimulator attachment is moveably connected to a movement means which moves the sexual stimulator attachment. A control arm is moveably connected to the housing and is in communication with the sexual stimulator attachment and moves the sexual stimulator attachment in response to movements of the control arm. A control means remotely controls the network enabled sexual apparatus by being in communication with the control means. In one embodiment, a live signal is buffered and used to augment a background control function to overcome network lag. Another embodiment includes a display to allow a user to view remotes users and to interact with them.

In one embodiment, a webcam is used to transmit video to enhance the interactivity therein. The apparatus is foldable for travel and is operable in both a horizontal and vertical orientation. Multiple users may access the apparatus in one embodiment. One embodiment includes mechanical linkages to provide the desired movement. Another embodiment utilizes servo motors to provide movement.

Other features and advantages of the instant invention will become apparent from the following description of the invention which refers to the accompanying drawings.

### BRIEF DESCRIPTION OF THE DRAWINGS

FIG. 1 is a perspective view of a network enabled sexual apparatus according to an embodiment of the present invention.

FIG. 2 is a perspective view of a network enabled sexual apparatus according to an embodiment of the present invention.

FIG. 3 is a perspective view of the network enabled sexual apparatus shown in FIG. 1.

FIG. 4 is a perspective view of the network enabled sexual apparatus shown in FIG. 1.

FIG. 5 is a perspective view of the network enabled sexual apparatus shown in FIG. 1 in a folded position.

FIG. 6 is a perspective view of the network enabled sexual apparatus shown in FIG. 1 in a vertical orientation.

FIG. 7 is a drawing illustrating the use of the network enabled sexual apparatus shown in FIG. 1.

FIG. 8 is a system diagram illustrating the use of a network enabled sexual apparatus connected to a communications network.

FIG. 9 is a system diagram illustrating the use of a network enabled sexual apparatus connected to a communications network with a plurality of users.

FIG. 10 is a perspective view of a network enabled sexual apparatus according to an embodiment of the present invention.

FIG. 11 is a system diagram illustrating the use of two network enabled sexual apparatus connected to a communications network.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

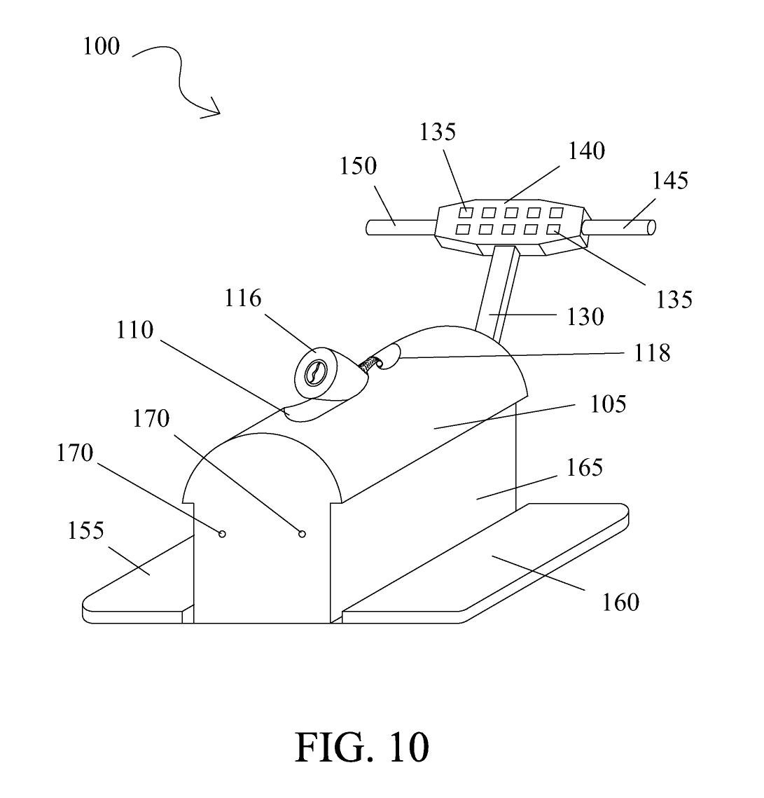

Referring to FIGS. 1 and 10, a network enabled sexual apparatus 100 is shown having a saddle portion 105 which is made of a compliant material such as Naugahyde ® or other material that is comfortable to sit on yet easy to clean. Additionally, a layer of foam or other cushioning material may be used to further enhance the user's comfort. An attachment opening 110 is formed in saddle portion 105 where attachments 115 and a male version 116 may be used depending on the kind of sexual stimulation the user wants. Various attachments may be used such as ribbed or textured dildos, realistic, slim, male masturbator, etc. Additionally, an attachment may be provided for anal stimulation if desired.

Attachments 115 and 116 have a connector at their base (not shown) that secures the attachments 115, 116 to the movement means (not shown). A clitoral stimulator 120 is provided to stimulate the clitoris of female user. Pressure and frequency can be adjusted as well as selecting a variable setting that randomly adjusts these settings. A housing 165 is used to provide structural support for saddle 105 and to house associated controls and communication network devices such a modems or WiFi interface. Clitoral stimulator 120 moves back and forth through openings 110 and 118.

A control arm 130 is hingedly attached to housing 165 and has a control panel 140 which has various controls 135 mounted thereon. Control 135 can control frequency of movement, type of movement (i.e. reciprocal, linear, vibrating, penetration depth, etc) associated with attachment 115 and clitoral stimulator 120. A left hand control 150 and a right hand control 145 may also be used to control speed and depth, etc. As a user moves control arm 130 in 3 degrees of freedom, attachment 115 may be manipulated in response to the movement. The movement may be achieved electronically using servos and wires or may be a mechanical linkage actually connected to control arm 130. In the mechanical embodiment, a series of pulleys, cables and biasing springs are used to control the motion of attachment 115.

In an electronic embodiment, a sensor(s) is used to coordinate the motion of control arm 130 with the movement of attachment 115. The movement in the electronic embodiment is provided through the use of servos motors in electrical communication with the sensor(s). A computer may be used to coordinate the motion. The sensors work in combination with computer to provide a feedback loop that is fed back in to the computer to enhance the user's experience. Additionally, most networks experience delays, dropped packets or other noise generally referred to as lag. In texting situations or even with voice conversations, the lag may be noticeable, but it is generally ignorable. Lagging might occur at inopportune times leading to frustration and a less than satisfying experience. For this reason, using feedback and an algorithm programmed to run on the computer, lag is masked by storing signals for a set period of time, for example one minute, before beginning to execute and refreshing the storage so that lag is unnoticed.

In one embodiment, the user can set the timing to compensate for a “noisy, slow or fast” network connection in order to optimize and enhance the user experience. Additional setting could allow a user to tailor program to “fill in” any missing feedback with a sample taken for a given amount of time to provide a consistent experience. As an example, if a first user enjoys slow, steady stimulation, the program would sample the last two minutes of use and fill in any gaps caused by lag with a similar experience. A second user might prefer more dynamic motions and the computer would fill in gaps with based on that preference.

In another embodiment, biometric sensors are used to gather response information from a user which is fed to the computer. The computer interprets the data and adjusts the stimulation attachments accordingly. For example, a sensor reads heart rate and pulse and determines that the stimulation needs to increase, but if the biological indicators show a lessened response the stimulation is adjusted and the sensor data is fed back to further adjust the stimulation. The algorithm that the computer runs is always driving the stimulation to produce the maximum stimulation in a feedback loop.

Examples of biological indicators that can be used to determine stimulation include, (but not meant to be exhaustive) pulse, heart rate, breathing rate, pupil dilation, body temperature, muscle activity, blood volume, genital temperature, and erection in males or swelling of clitoris in females. There are many sensors available for measuring these responses ranging from wearable undergarments to various probes and contacts. Wearable accelerometers can be used to wireless transmit muscle position and movement to the computer.

Additionally, in one embodiment, the user chooses a setting that provides a continuous “background” level of stimulation where the networked user or users input is fed on top of the background to provide additional stimulation and interaction. In this embodiment, any lag is unnoticeable due to the background operation. The background operation is not necessarily constant. Various “background” settings are user selectable to adapt the experience based on the user's preference.

A left folding wing 155 and a right folding wing 160 provide stability when deployed as shown. Two saddle extension supports 170 are provide to attach optional saddle extension 215 (FIG. 3).

Referring now to FIG. 2, a network enabled sexual apparatus 200 is shown having a DVD player or drive 185 built in and disposed within housing 165. A monitor 175 is attached to control arm 130 along with an input device 180 such as a keyboard with trackball built in. Of course other kind of input devices may be used including voice operated and other sensory detecting devices like head mounted control input, etc. A webcam 270 is used to transmit video data across a communication network like the World Wide Web or Web 2.0, etc. A computer (not shown) is disposed within housing 165 to control operation of device 200.

A series of computer interfaces 190 are provided to attach various devices therein. USB and Firewire are two popular interfaces, but as others become available, they would be acceptable to provide access to the processor. Audio and video interfaces 195 may also be provided for additional operation. A telephone connector 205 and network connection 210 are provided for network connectivity as well as a wireless capability as is known in the art. The DVD player may be used to view material such as adult movies, or personal messages recorded by a loved one.

Referring now to FIGS. 3 and 4, a saddle extension 215 is used to extend the saddle area to accommodate two people or just to give more room to a single user. Saddle extension 215 fits in extension supports 170 and may be secured using nut or wing nut. Additionally, other kind of connectors may be used such as snap rings, keyed supports etc. as is known in the art as long as saddle extension 215 is securely held in place during use, but remains removable.

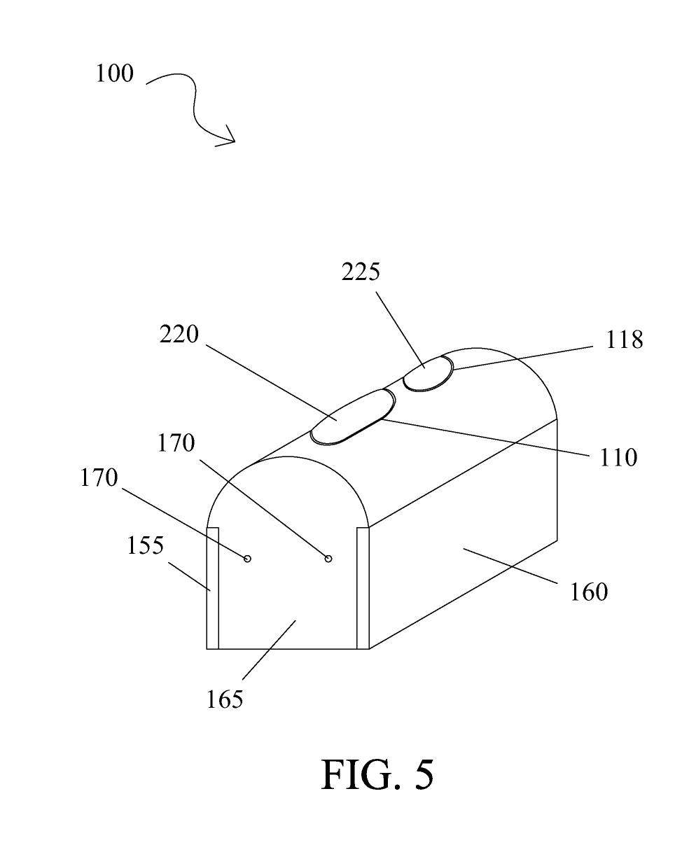

Referring to FIG. 5, network enabled sexual apparatus 100 is shown in a folded up configuration. Left folding wing 155 and right folding wing 160 are folded up to fit within a recessed portion of housing 165. Opening covers 220 and 225 respectively slide over openings to protect the contents. A handle (not shown) may be provided to facilitate carrying.

Now referring to FIG. 6, network enabled sexual apparatus 100 is shown in a vertical orientation. In this configuration, a user can lie down and straddle device aligning attachment 115 and clitoral stimulator 120 to achieve the desired sexual pleasure. Control arm 130 is shown rotating around hinge 250 and fitting within recessed portion 235 to achieve the compact configuration shown in FIG. 5. To stabilize device 100 while in the vertical orientation, two legs 230 may be rotated around hinges 255 to increase the base footprint. Legs 230 fit within recessed portions 240 when not in use. A control arm actuator 245 is used to translate control arm 130 movement into movements of attachment 115 and or clitoral stimulator 120. Again, this may be achieved using mechanical linkages such as cables, springs and pulleys or electronically using sensors as is known in the art.

Referring to FIG. 7, a user 260 straddles saddle portion 105 and aligns attachment and clitoral stimulator to achieve the desired level of sexual stimulation. Of course other adjustment means may be used as long attachment and clitoral stimulator can be positioned to accommodate various body types.

FIGS. 8 and 9 illustrate the network enabled embodiment where network enabled sexual apparatus 200 is shown connected to a communications network 280 such as the world wide web. Connection 275 may be wired, WiFi, LAN, Ethernet, WAN or other connectivity scheme to provide access to a network. A network interface device 285 is used to connect to network 280. Network interface device 285 may be a personal computer, Blackberry device, web-enabled telephonic device or other network enabled interface as is known in the art. Once both devices are connected, a user accessing network enabled device 200 is able to remotely control attachment and clitoral stimulator as well as view the activity via webcam. The user may connect via a dedicated or a commercially operated site where access is sold on pricing arrangement such as access by the minute, monthly subscription, etc. Multiple users are accommodated in the configuration shown in FIG. 9 where a plurality of users can participate in a group sexual activity by agreeing to a pricing plan to provide access.

In addition to pay sites, this device may be used to enhance relationships with people who are geographically located apart such as deployed soldiers, spouses show find themselves traveling for business, etc. The present invention provides a level of interactivity not available by tradition methods such as “phone sex” etc.

Referring to FIG. 11, two network enabled sexual apparatus 200 are shown connected through communications network 280 using connections 275. This configuration allows total user interaction with each user being virtually aware of the other.

Although the instant invention has been described in relation to particular embodiments thereof, many other variations and modifications and other uses will become apparent to those skilled in the art.

What is claimed is:

1. A network enabled sexual apparatus with feedback and fault tolerance comprising:
a saddle portion;
a housing supporting said saddle portion;
a sexual stimulator attachment disposed on an exterior top portion of said saddle portion;
said sexual stimulator being adapted to respond to a control signal;
a processing means for generating said control signal; and
said processing means also for interfacing with a network.

2. The network enabled sexual apparatus with feedback and fault tolerance according to claim 1 wherein said control signal is at least partially formed in response to at least one input signal transmitted by said network.

3. The network enabled sexual apparatus with feedback and fault tolerance according to claim 1 wherein said processing means is a computer.

4. The network enabled sexual apparatus with feedback and fault tolerance according to claim 3 further comprising a program adapted to run within said computer whereby said control signal is customizable.

5. The network enabled sexual apparatus with feedback and fault tolerance according to claim 3 wherein said control signal is first stored in a buffer before being sent to said sexual stimulator.

6. The network enabled sexual apparatus with feedback and fault tolerance according to claim 1 wherein said sexual stimulator is adapted to stimulate at least one female erogenous zone.

7. The network enabled sexual apparatus with feedback and fault tolerance according to claim 1 wherein said sexual stimulator is adapted to stimulate at least one male erogenous zone.

8. The network enabled sexual apparatus with feedback and fault tolerance according to claim 1 wherein said sexual stimulator is user selectable to stimulate at least one erogenous zone.

9. The network enabled sexual apparatus with feedback and fault tolerance according to claim 1 wherein said sexual stimulator comprises a vibrating mechanism.

10. The network enabled sexual apparatus with feedback and fault tolerance according to claim 1 wherein said sexual stimulator comprises a movable mechanism.

11. The network enabled sexual apparatus with feedback and fault tolerance according to claim 1 wherein said sexual stimulator comprises a multifunctional mechanism whereby different sensations are produced in response to said control signal.

12. The network enabled sexual apparatus with feedback and fault tolerance according to claim 11 wherein said multifunctional mechanism is adapted to produce at least one of a thrusting, vibrating and rotating motion.

13. The network enabled sexual apparatus with feedback and fault tolerance according to claim 1 further comprising a pair of stabilizing legs rotatably attached to said housing whereby said housing may be placed in a vertical orientation.

14. The network enabled sexual apparatus with feedback and fault tolerance according to claim 1 further comprising a hand control portion connected to said housing wherein a user controls at least one control function.

15. The network enabled sexual apparatus with feedback and fault tolerance according to claim 14 further comprising a display operably connected to said housing wherein said user can view a selected image.

16. The network enabled sexual apparatus with feedback and fault tolerance according to claim 15 wherein said selected image includes at least a live image of a remote user.

17. The network enabled sexual apparatus with feedback and fault tolerance according to claim 15 further comprising an audio means adapted to allow a user to auditorially interact with said remote user.

18. The network enabled sexual apparatus with feedback and fault tolerance according to claim 15 further comprising a web camera adapted to send an image of said user to said network.

19. The network enabled sexual apparatus with feedback and fault tolerance according to claim 4 further comprising at least one sensor means for sensing said user' biological response to sexual stimulation and feeding that data back to said computer.

20. The network enabled sexual apparatus with feedback and fault tolerance according to claim 19 wherein said control signal is adjusted in response to said data to cause further stimulation.
